(19) United States
(12) Patent Application Publication
(10) Pub. No.: US 2011/0217764 A1
(43) Pub. Date: Sep. 8, 2011

(54) Rotating Bioreactor and Spool Harvester Apparatus for Biomass Production

(75) Inventors: Logan Christenson, Logan, UT (US); Ronald Sims, Logan, UT (US)

(73) Assignee: UTAH STATE UNIVERSITY, North Logan, UT (US)

(21) Appl. No.: 13/040,364

(22) Filed: Mar. 4, 2011

Related U.S. Application Data
(60) Provisional application No. 61/310,360, filed on Mar. 4, 2010.

(51) Int. Cl. C12M 1/10

(52) U.S. Cl. 435/289.1

(57) ABSTRACT

An apparatus that exposes a biofilm growth surface to liquid media as it rotates. A biofilm growth substratum is wound around a rotatable body in the form of a non-rigid material capable of supporting biofilm growth. A harvester receives the biofilm laden substratum, collects the biofilm as a biomass and reloads the substratum onto the rotatable body.

### RELATED APPLICATIONS

This application claims the benefit under 35 U.S.C. §119(e) of U.S. Provisional Patent Application No. 61/310,360 filed Mar. 4, 2010, and titled “Biomass production using a rotation bioreactor and spool harvester” which is incorporated herein by reference.

### FIELD OF THE INVENTION

This technology is an apparatus for growing and harvesting biomass for use as feedstock in, for example, the production of products or in wastewater remediation.

### BACKGROUND

Excess nitrogen, phosphorus, and other nutrients or compounds in discharged wastewaters can lead to downstream eutrophication and ecosystem damage. Advanced wastewater treatment technologies capable of removing these nutrients are expensive and often require the addition of chemical precipitants. Nitrogen and phosphorus can be removed naturally through biomass assimilation, but heterotrophic bacteria typically become carbon limited before removing all soluble N and P. Because microalgae are autotrophic, they can overcome this limitation and assimilate the remaining nutrients. In addition to the environmental benefits of harvesting algae grown during wastewater treatment, harvested microalgae are valuable as fertilizer, high-protein animal feed, and feedstock for the production of biofuels, including biodiesel and biomethane. Nutraceuticals, polymers, and other valuable products can be obtained from microalgae as well. Previously however, the realization of such benefits has been handicapped by an inability to find a reliable and cost effective apparatus and method of growing and harvesting the algae.

Previous methods of growing algae at large scale include open outdoor pond systems and closed tubular photobioreactors. The most common outdoor pond design is the high rate algal pond, or raceway pond. These are shallow ponds that circle a volume of nutrient rich water by means of a paddle wheel. Although relatively inexpensive to build, large plots of land are required and the resulting algae yields are lower than with closed reactors. Tubular photobioreactors can often achieve higher cell concentrations than open ponds, but suffer from high material cost and frequent cell death due to inefficient gas exchange. Biofouling of the reactor walls also decreases light penetration and cleaning becomes an issue as well. With both methods, the resulting solution of suspended microalgae is very dilute, necessitating high cost methods of separation.

Suspended microalgae must be removed from very dilute solutions and concentrated before further processing is possible. Current separation methods include filtration, sedimentation, centrifugation, dissolved air flotation, addition of electrolytes and polymers to induce coagulation and flocculation, and multiple combinations of these operations. Separation through filtration is difficult due to the small size of planktonic microalgae, and the sedimentation rate of algae is too slow for separation on a reasonable time scale. Dissolved air flotation requires high energy and high electrolyte and/or polymer addition to sufficiently flocculate microalgae. Centrifugation is currently the most common method used to separate algae from aqueous solutions; however, high upfront capital costs, power demand, and frequent maintenance make it uneconomical for large scale use.

In addition to planktonic growth, microalgae are also capable of growing as biofilms attached to surfaces. Algal biofilms, or periphyton, are able to remove nutrients from wastewater just as suspended algae, and harvested biofilms can be processed into valuable products just as harvested suspended algae. When algae are grown as biofilms, the biomass is naturally concentrated and more easily harvested, leading to more direct removal and reduced downstream processing. The extracellular polymeric substance secreted by biofilms also increases the flocculation of associated suspended cells. Previously, however, there were no methods of growing and harvesting algal biofilms with any full scale potential.

In addition to microalgae, other microorganisms are capable of growing as biofilms attached to surfaces. Biofilms are often complex mixed cultures containing microalgae, cyanobacteria, heterotrophic bacteria, nitrifying bacteria, microscopic fungi, and various combinations of these types of organisms. When grown as biofilms, the organism's morphology and metabolism are often different than when the organism is suspended. These changes are often beneficial, and can include increased production of a desired product. Biofilm reactors designed for the purpose of growing attached cultures include continuous stirred tank reactors (CSTR) with fibrous bed support, biofilm packed bed reactors (BPBR), biofilm trickling bed reactors (BTBR), and biofilm fluidized bed reactors (BFBR). Such reactors use a porous support or small granules as substrata for cell attachment and biofilm growth. These reactor configurations are often used to treat wastewater or produce a secreted product, but are limited in that harvesting of the biomass or intracellular product is not possible without high cost.

### SUMMARY

In one embodiment, we describe a reactor for the production of biomass involving a rotating cylinder or cylinders partially submerged in liquid media. The rotating cylinders are outfitted with a substratum capable of biofilm growth. The substratum is in a form that allows it to be wound around the cylinder, allowing the reactor to act as a spool and the harvesting of the biomass and reloading of the reactor are accomplished simultaneously.

### BRIEF DESCRIPTION OF THE ILLUSTRATIONS

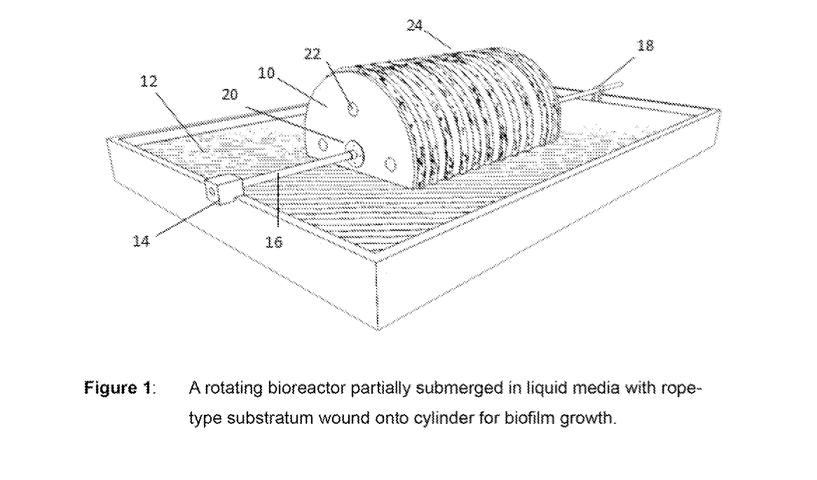

FIG. 1 shows a rotating bioreactor partially submerged in liquid media with rope type substratum wound onto cylinder for biofilm growth.

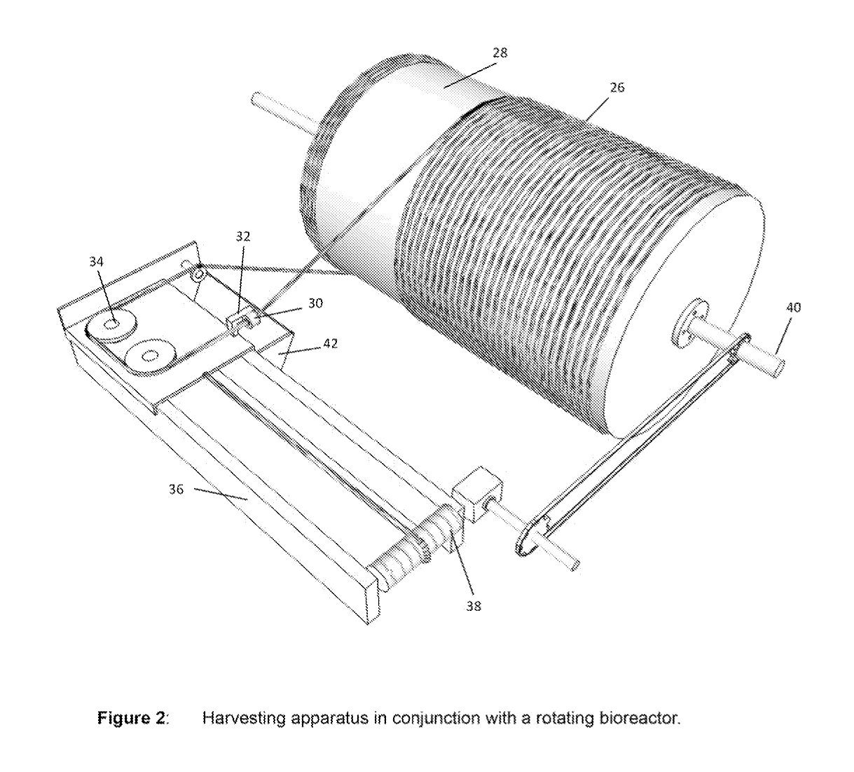

FIG. 2 shows a harvesting apparatus in conjunction with a rotating bioreactor.

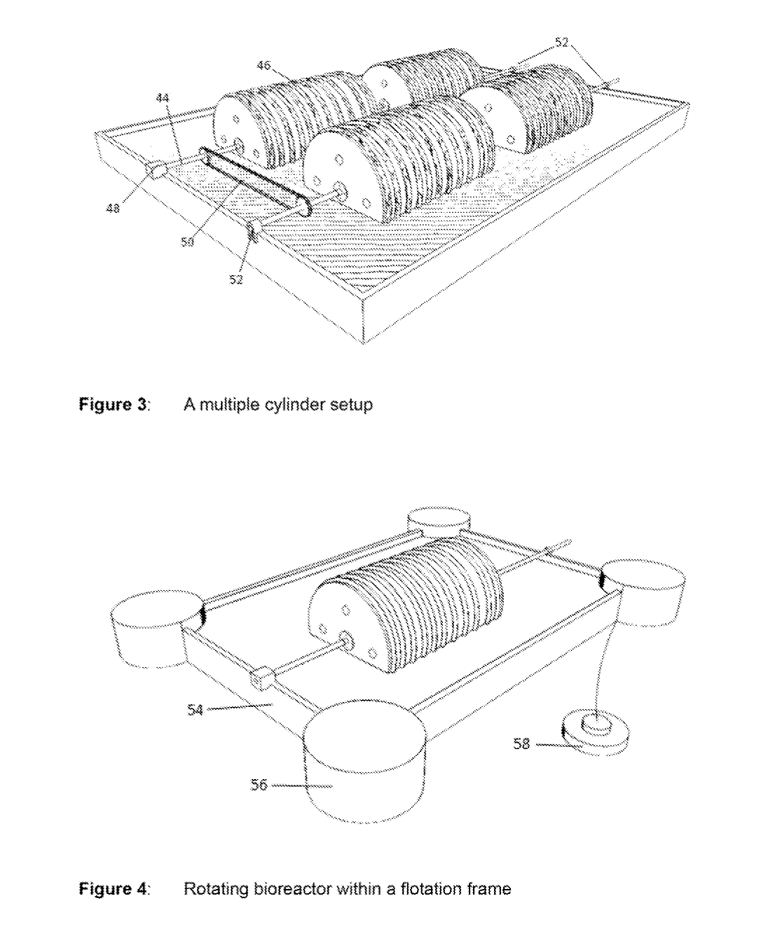

FIG. 3 shows a multiple cylinder setup.

FIG. 4 shows a rotating reactor within a flotation frame.

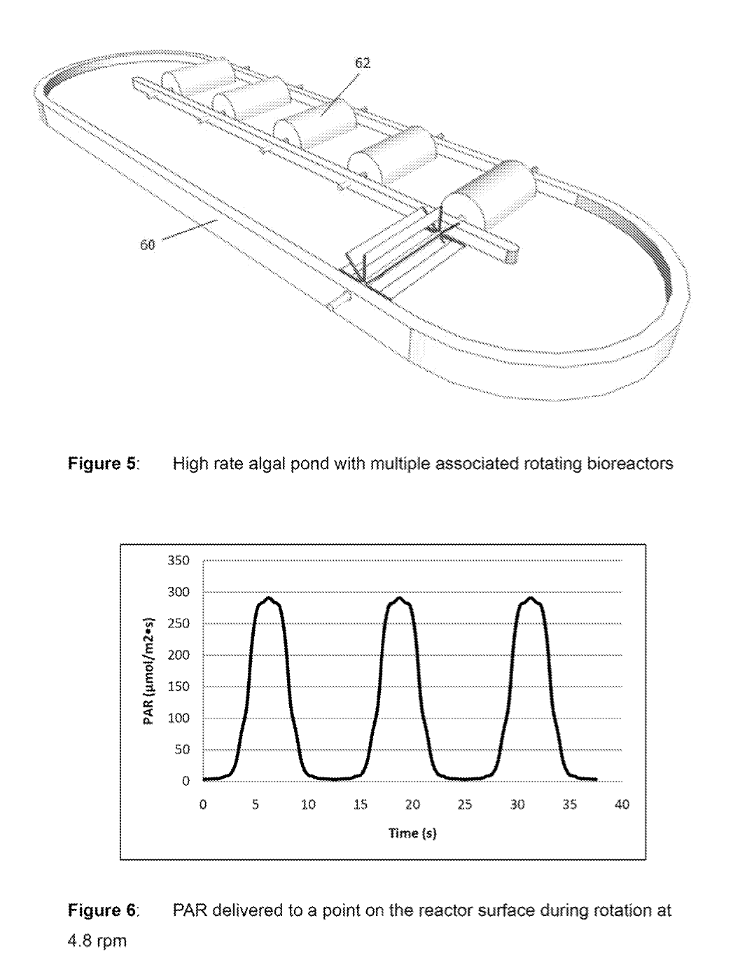

FIG. 5 shows a high rate algal pond with associated rotating bioreactors.

FIG. 6 shows the photosynthetically active radiation cycle of bench scale reactors when operated at 4.8 rpm.

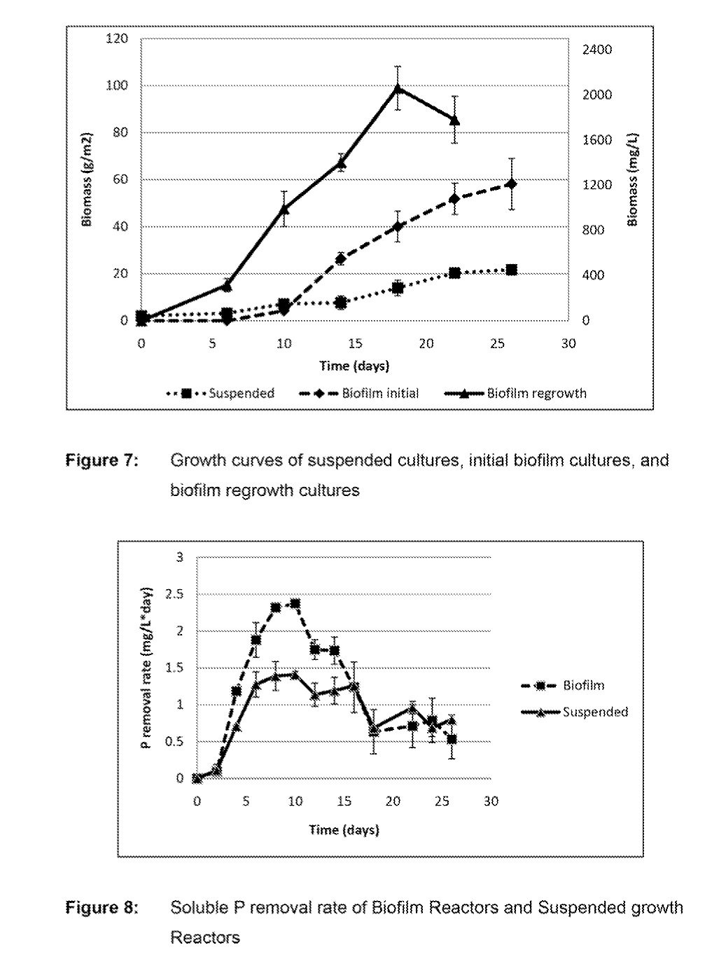

FIG. 7 shows the growth curves of a suspended culture, initial biofilm culture, and secondary biofilm culture.

FIG. 8 shows soluble P removal rates and soluble P concentrations of the suspended and biofilm reactors.

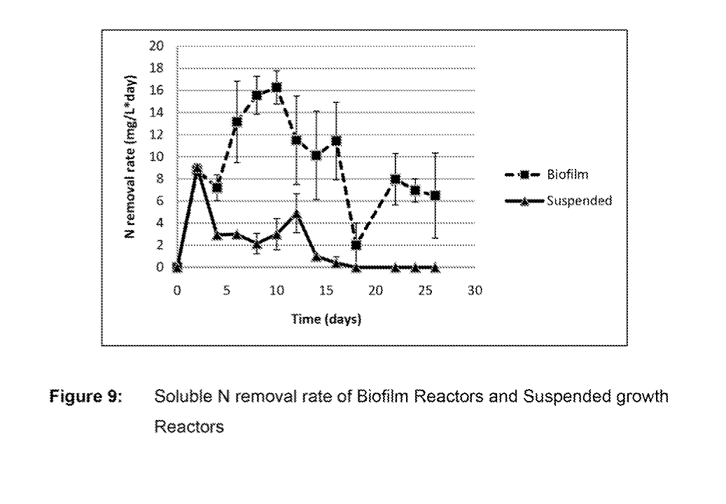

FIG. 9 shows soluble N removal rates and soluble N concentrations of the suspended and biofilm reactors.

### DETAILED DESCRIPTION OF THE INVENTION

In one embodiment, we describe a rotating bioreactor apparatus. In FIG. 1 there is shown a body 10 partially submerged in a liquid medium 12. In this embodiment the body is in the form of a right circular cylinder. Additional body formats may be utilized including, but not limited to, elliptic cylinder, parabolic cylinder, hyperbolic cylinder, generalized cylinder or oblique cylinder or any form with a rotational axis suitable for this purpose.

One skilled in the relevant art will recognize that different formulations of liquid medium 12 will be used to produce different types of biomass. The liquid medium 12 may be a complex, defined, or selective growth medium. More specifically, the liquid medium 12 may be a complex medium including, but not limited to complex dextrose based media, sea water media, domestic wastewater, municipal wastewater, industrial wastewater, surface runoff wastewater, soil extract media, or any natural water containing detectable amounts of phosphorus or nitrogen; or a defined medium, including, but not limited to Bristol's medium, Bolds Basal medium, Walne medium, Guillard's f medium, Blue-Green medium, D medium, DYIY medium, Jaworski's medium, K medium, MBL medium, Jorgensen's medium, and MLA medium; or a selective medium including, but not limited to minimal media based on specific nutrient auxotrophy, and selective media that incorporates antibiotics. Depending on the chosen liquid medium 12 and seed culture, the resulting biofilm may be a mixed or pure culture and may be comprised of microalgae, cyanobacteria, nitrifying bacteria, heterotrophic bacteria, microscopic fungi, or any combination thereof.

Still referring to FIG. 1, a rotation device 14 transmits rotational power to a drive shaft 16 that runs through the center of the cylinder 10 and is supported by a bearing 18 opposite the rotation device 14. Where the drive shaft 16 enters and exits the cylinder 10, a base plate 20 is used to connect the drive shaft 16 and the cylinder 10. Holes 22 are made in the ends of the cylinder 10 to allow liquid media 12 to enter the cylinder 10. A substratum 24 is placed around the cylinder 10 for biofilm growth.

In more detail, still referring to FIG. 1, the rotation device 14 transmits rotational power to the drive shaft 16, causing the cylinder 10 to rotate with the drive shaft 16. As the cylinder 10 rotates, the biofilm substratum 24 placed on the surface of the cylinder 10 is alternately exposed to the liquid media 12 and the air.

In further detail, still referring to FIG. 1, the biofilm substratum 24 may be in the form of a rope, cable or belt or the like such that it can be wound around the outer circumference of the cylinder 10. The substratum 24 may be selected from a group comprising cotton, jute, hemp, manila, silk, linen, sisal, silica, acrylic, polyester, nylon, polypropylene, polyethylene, polytetrafluoroethylene, polymethylmethacrylate, polystyrene, polyvinyl chloride, or any other non-rigid material capable of supporting biofilm growth. One end of the substratum 24 is attached to one end of the surface of the cylinder 10 and wound around until the surface of the cylinder 10 may be sufficiently covered with the substratum 24. The free end of the substratum 24 may then be attached to the surface of the cylinder 10 to keep the substratum 24 from unwinding during rotation of the cylinder 10.

In another embodiment, we describe a harvesting apparatus in conjunction with a rotating bioreactor. Referring now to FIG. 2, the biofilm is collected by detaching one end of the substratum 26 from the cylinder 28 and threading it through a scraper 30. The scraper 30 may be a blade, series of blades, simple piece of rigid material with a hole in it, or more preferably, a unit with an adjustable diameter and/or constant tension settings like a hose clamp. The scraper 30 may be held in place by attachment to a support 32. A reorientation system 34 is provided to prevent twisting or binding of the substratum 26. The loose end of the substratum 26 is threaded through the scraper 30 and reorientation system 34 until it can be reattached to the cylinder 28. As the cylinder 28 continues to rotate, the entire length of the substratum 26 may be pulled through the scraper 30 and pulley system 34 and rewound onto the cylinder 28. To ensure the substratum 26 may be properly rewound onto the entire length of the cylinder 28, the scraper 30, support 32, and pulley system 34, are pulled on a support frame 36 along the length of the cylinder 28 at a rate such that the harvested portion of the substratum 26 may not be layered on top of itself as it is rewound. This may be accomplished with a lateral movement system 38 that may be powered by connection to the drive shaft 40 powering the cylinder 28. Appropriate gear ratios may be chosen to achieve the desired pull rate and spacing of substratum 26. As the biofilm is removed from the substratum 26, it is gathered in a collection bin 42.

Referring now to another embodiment describing a multiple cylinder setup, shown in FIG. 3, a drive shaft 44 may be made long enough to support two or more cylinders 46 in a train formation. More cylinders 46 may be placed so that rotational power from a motor 48 is transferred to two or more drive shafts 44 through a power transfer mechanism like a roller chain 50. The drive shafts 44 are supported by bearings 52 on each end.

Referring to another embodiment shown in FIG. 4, the entire apparatus may be placed within a support frame 54 with attached floats 56. The apparatus can then be placed at a suitable site and held in place using an anchor 58 or other suitable means of holding it in place. One application of this embodiment of the invention is a retrofitting of oxidation lagoons at a wastewater treatment plant.

Referring to FIG. 5, another embodiment places the apparatus with a high rate algae pond 60 like a raceway or meandering ditch. The cylinders 62 may be rotated by the force of the passing water or powered by a motor and shaft connected to the cylinder. In a further embodiment, the cylinders 62 may be rotated by an air supply directed at the submerged perimeter of the cylinder in a direction perpendicular to the axis of rotation. In embodiments such as this, the biofilm enhances flocculation of the suspended culture, leading to inexpensive harvesting of all the biomass in the system.

### EXAMPLES

### Example 1

In one embodiment, several bench scale units of the type shown in FIG. 1 were used with 8 liters of chlorinated weak domestic strength wastewater as seeding media. A nested factorial experiment with triplicate replication of samples was established to determine the most suitable substrata for biofilm growth. The initial total suspended solids content of the wastewater was 42 mg/l. Concentrations of soluble phosphorus and nitrogen were brought to 5 mg/l and 36 mg/l respectively using KH₂PO₄, K₂HPO₄, and NaNO₃. As a fed batch operation, N and P were added every 48 hours to give an average total P of 5.0 mg/l, and an average total N of 52.7 mg/l. Soluble N and P averaged 26.2 mg/l and 3.7 mg/l, respectively. A light cycle of 14 hours on, 10 hours off was used throughout the experiment. FIG. 6 shows the cycle of photosynthetically active radiation (PAR) delivered to a point on the reactor during rotation at 4.8 rpm during periods while the lights were on. Biomass was harvested after 22 days of growth. This time included a recovery period following chlorination. Table 1 summarizes the results on the basis of mass per liquid surface area.

TABLE 1
Avg. Biomass yield of different substrate materials

| | Avg. Biomass | |
|---|---|---|
| | Yield | Std. |
| Substrata | (g/m²) | Deviation |
| Cotton Rope | 91.2 | 10.4 |
| Cotton (High thread | 62.2 | 0.9 |
| count) | | |
| Jute | 51.4 | 5.1 |
| Cotton (Low thread | 51.3 | 1.9 |
| count) | | |
| Acrylic | 49.3 | 0.4 |
| Polyester | 19.3 | 1.8 |
| Polypropylene | 0 | 0 |
| Nylon | 0 | 0 |
| Construction Paper* | 0 | N/A |
| Sisal* | 0 | N/A |
| Lignin based cover* | 0 | N/A |

*Materials showed some growth but biomass was not harvestable

The substrata that were placed onto the cylinder as a sheet were harvested using a simple scraper blade. This proved to be difficult due to the constant adjustments required to scrape the uneven biofilm growth. Such substrata had also loosened during reactor operation causing frequent snagging and tearing against the scraper blade and rendering the materials unsuitable for future use. Cotton rope gave the highest biomass yields, and the rope construction allowed application of the harvesting method shown in FIG. 2. The cotton rope incurred no damage during harvesting and was immediately reused.

### Example 2

In another embodiment, the same procedure described in Example 1 was repeated with cotton rope as the only substratum. Triplicate samples were harvested after 10, 14, 18, 22, and 26 days of growth. Suspended cultures were also grown in reactor tanks of the same dimensions with the same light and nutrient conditions as the biofilm reactors. The same weak domestic strength wastewater was used to seed each type of reactor. Power input for mixing the suspended cultures was the same as the power input for rotating the cylinders. After each biofilm harvest, the substrata were reloaded onto the reactor to determine the secondary growth curve. Regrowth samples were harvested after 6, 10, 14, 18, and 22 days of growth. Growth in the suspended culture reactors was determined using the glass fiber filter method. FIG. 7 shows the growth curves of the initial biofilms, secondary biofilms, and suspended cultures. It can be seen that the biofilm grows at a much faster rate after the initial harvest. This is most likely due to the residual biomass left on the substratum after scraping. This secondary growth curve represents the true productivity of the reactor when operated continuously. Table 2 compares the maximum productivity obtained by each type of growth and at what time it was obtained.

TABLE 2
Maximum productivity obtained by different growth types

| | Yield* | Time | Productivity* |
|---|---|---|---|
| Growth Type | (g/m²) | (days) | (g/m² day) |
| Biofilm initial | 51.6 ± 6.6 | 22 | 2.4 ± 0.3 |
| Biofilm | 98.9 ± 9.3 | 18 | 5.5 ± 0.5 |
| regrowth | | | |
| Suspended | 20.4 ± 1.4 | 22 | 0.9 ± 0.1 |

*plus and minus one standard deviation from the mean

### Example 3

In another embodiment, nitrogen and phosphorus concentration data from the experiment of Example 2 were analyzed to determine the wastewater remediation ability of the suspended culture and the biofilms. After filtration of wastewater samples, soluble N concentrations were determined using the chromotropic acid method for nitrate-N and the salicylate method for ammonia-N. Soluble P as orthophosphate was determined using the ascorbic acid method. The wastewater samples were also analyzed for total N and P using the chromotropic acid method with alkaline persulfate digestion and the molybdovanadate method with acid persulfate digestion, respectively.

FIG. 8 shows soluble P removal rates for the suspended and biofilm cultures. FIG. 9 shows soluble N removal rates for the suspended and biofilm cultures. It can be seen that the biofilm reactors demonstrated higher removal of both nitrogen and phosphorus compared to the suspended culture reactors. Furthermore, these nutrients could be easily removed from the system by simply removing the biofilm as shown in FIG. 2, whereas the suspended cultures would have to be removed through centrifugation, filtration, or the like to completely remove the nutrients from the system.

### Example 4

In another embodiment, as the biofilms of the experiments of Example 1 and Example 2 were grown, a visual observation of the wastewater turbidity was made for each tank containing a rotating bioreactor. It was observed that at some point during operation, typically between 12-18 days of growth, the suspended microorganisms in the wastewater associated with the rotating bioreactors underwent spontaneous autoflocculation and settled to the bottom or floated to the top of the reactor tank. Such flocculated biomass would be much easier to harvest than a suspended culture.

From the foregoing description, one skilled in the art can easily ascertain the essential characteristics of this invention, and without departing from the spirit and scope thereof, can make changes and modifications of the invention to adapt it to various usage and conditions. The preceding preferred specific embodiments are to be construed as merely illustrative, and not limiting of the scope of the invention in any way whatsoever.

What is claimed is:

1. An apparatus comprising:
a rotatable body contacting a liquid media;
said rotatable body having a first outer surface which is approximately parallel to an axis of rotation;
a removable substratum wound around said first outer surface of said rotatable body;
said substratum configured to support growth of a microorganism biofilm; and
a harvesting device configured to harvest said biofilm from said substratum.

2. The apparatus of claim 1 wherein said substratum is passed to said harvesting device wherein said biofilm is harvested from said substratum and gathered in a collection bin.

3. The apparatus of claim 1 wherein said harvested substratum is rewound around the first outer surface of said rotating body.

4. The apparatus of claim 1 wherein said rotatable body is partially submerged in said liquid media.

5. The apparatus of claim 1 wherein said rotating body is a generalized cylinder.

6. The apparatus of claim 5 wherein said rotating body is a right circular cylinder.

7. The apparatus of claim 5 wherein said rotating body is an elliptic cylinder.

8. The apparatus of claim 1 wherein said substratum passes through a scraper mechanism to extract said biofilm from said substratum.

9. The apparatus of claim 1 wherein said substratum is a non-rigid material capable of supporting biofilm growth.

10. The apparatus of claim 9 wherein said substratum is a rope.

11. The apparatus of claim 9 wherein said substratum is a belt.

12. The apparatus of claim 9 wherein said substratum is a cable.

13. The apparatus of claim 9 wherein the said substratum composition is selected from a group consisting of: cotton, jute, hemp, manila, silk, linen, sisal, silica, acrylic, polyester, nylon, polypropylene, polyethylene, polytetrafluoroethylene, polymethylmethacrylate, polystyrene and polyvinyl chloride.

14. The apparatus of claim 1 wherein said harvesting device comprises a ring shaped scraper.

15. The apparatus of claim 14 wherein said ring shaped scraper has an adjustable diameter.

16. The apparatus of claim 14, wherein said ring shaped scraper induces a constant tension during contact with said substratum.

17. The apparatus of claim 1 wherein said harvesting device comprises a scraper blade.

18. The apparatus of claim 16 wherein said scraper blade has adjustable positioning.

19. The apparatus of claim 17, wherein said scraper blade induces a constant tension during contact with said substratum.

20. The apparatus of claim 1, wherein said liquid media is a growth medium capable of supporting growth of a microorganism.

21. The apparatus of claim 20 wherein growth medium is selected from a group consisting of: Bristol's medium, Bolds Basal medium, Walne medium, Guillard's f medium, Blue-Green medium, D medium, DYIY medium, Jaworski's medium, K medium, MBL medium, Jorgensen's medium, and MLA medium.

22. The apparatus of claim 20, wherein the said liquid media is a selective media selected from a group consisting of: minimal media based on specific nutrient auxotrophy and selective media that incorporates antibiotics.

23. The apparatus of claim 20, wherein the said liquid medium is a complex medium selected from a group consisting of: complex dextrose based media, sea water media, soil extract media, domestic wastewater, municipal wastewater, industrial wastewater, surface runoff wastewater and naturally occurring waters containing detectable levels of nitrogen or phosphorus.

24. The apparatus of claim 1, wherein said biofilm has remnant residuals remaining attached to said substratum to seed regrowth after passing through said harvesting device.

25. The apparatus of claim 1, further comprising:
a lateral movement system;
said lateral movement system movable approximately parallel to the rotational axis of said rotatable body; and
said harvester connected to said lateral movement system, wherein said harvester moves along the length of said rotatable body as said substratum is rewound onto said rotatable body.

26. The apparatus of claim 1 wherein said rotatable body is partially exposed to air.
